(19) United States
(12) Patent Application Publication
(10) Pub. No.: US 2026/0051055 A1
(43) Pub. Date: Feb. 19, 2026

(54) DOMAIN SWAP AND ARTIFICIAL GENERATED VIRTUAL IMAGES

(71) Applicant: VENTANA MEDICAL SYSTEMS, INC., Tucson, AZ (US)

(72) Inventors: Qiangqiang Gu, Pleasanton, CA (US); Hauke Kolster, Pleasanton, CA (US); Matthew Olson, Pleasanton, CA (US); Xingwei Wang, Tucson, AZ (US); Zuo Zhao, Pleasanton, CA (US)

(73) Assignee: VENTANA MEDICAL SYSTEMS, INC., Tucson, AZ (US)

(21) Appl. No.: 19/368,630

(22) Filed: Oct. 24, 2025

Related U.S. Application Data
(63) Continuation of application No. PCT/US2024/026631, filed on Apr. 26, 2024.
(60) Provisional application No. 63/499,083, filed on Apr. 28, 2023.

(51) Int. Cl. G06T 7/00; G06N 3/0475; G06T 7/11; G16H 30/40

(52) U.S. Cl. CPC G06T 7/0012; G06N 3/0475; G06T 7/11; G16H 30/40; G06T 2207/10088; G06T 2207/30096

(57) ABSTRACT

The present disclosure relates to domain swap by accessing an image from a first domain and is processed using a machine learning model to generate a virtual synthetic image in a second domain. This approach can eliminate or reduce the need to separately collect an image in the second domain, which can save time and cost. Leveraging tools that are available in the second domain to perform image processing on the virtual synthetic image. Results or analysis from the image processing in the second domain can then be directly applied to the first domain and to assess the image further. Since spatial reference points (size, scale, view etc.) are same, pixels identifying a boundary of a region depicted in the virtual synthetic image are the same pixels in the first domain.

### CROSS-REFERENCE TO RELATED APPLICATIONS

This application is a continuation of PCT Patent Application No. PCT/US2024/026631, filed on Apr. 26, 2024, which claims the priority to and the benefit of U.S. Provisional Application No. 63/499,083, filed on Apr. 28, 2023, entitled “Domain Swap and Artificial Generated Virtual Images”. The entire disclosures of the aforementioned applications are incorporated by reference herein in their entireties for all purposes.

### BACKGROUND

Digital pathology may involve the interpretation of digitized images in order to correctly diagnose subjects and guide therapeutic decision making. In digital pathology solutions, image-analysis workflows can be established to automatically detect or classify biological objects of interest e.g., positive, negative tumor cells, etc. An exemplary digital pathology solution workflow includes obtaining tissue slides, scanning preselected areas or the entirety of the tissue slides with a digital image scanner (e.g., a whole slide image (WSI) scanner) to obtain digital images, performing image analysis on the digital image using one or more image analysis algorithms, and potentially detecting, quantifying (e.g., counting or identify object-specific or cumulative areas of) each object of interest based on the image analysis (e.g., quantitative or semi-quantitative scoring such as positive, negative, medium, weak, etc.).

Digital pathology may use singleplex or multiplex techniques. Singleplex uses a stain for just one biomarker and also a reference stain. Meanwhile, multiplex or MPX involves the staining for two or more biomarkers (in addition to the reference stain) in a single slide. Therefore, multiplex techniques support simultaneous detection of multiple biomarkers and their co-expression at a single-cell level. However, it is challenging for pathologists to annotate tumors in multiplex slides. Cross-validation among biomarkers involves a comparison of biomarker status against morphological features. The morphological features can be identified by using another stain, such as with hematoxylin and eosin (H&E). H&E is absorbed by nuclei, the extracellular matrix and the cytoplasm.

A multiplex slide itself cannot be stained chemically with H&E, as absorption of H&E may make it difficult or impossible to reliably and precisely detect biomarker signals. Therefore, workflows have traditionally involved staining a section that is adjacent to a multiplex section with H&E (See FIG. 1). However, this results in a need for extra slides (the H&E slides) to be prepared and annotated manually by a pathologist, which results in extra time, extra use of glass, cost and labor and also requires that more tissue be collected for the assessment. A traditional multiplex analysis requires a spatial registration to make analysis results from an H&E serial section to be used on multiplex image. However, this labor-intensive spatial registration process is never fully accurate and artifacts on either slide can ruin the analysis. This is because, a section is frequently 4 microns thick, while a cell is frequently 4-20 microns diameter (and potentially quite asymmetric), meaning that a given cell might not be visible on two adjacent slides.

Further, to develop multiplex algorithms, multiplex image analysis requires the use of registered H&E images to confirm, for example, segmentation of tumor or stroma. The multiplex analysis requires a cross validation of multiple biomarkers in context of H&E morphology. Hence, multiplex algorithm development is very complex and requires a high registration process that cannot be fully accurate. Therefore, it would be advantageous if reference features could be accurately and reliably identified in a manner that requires less tissue, time, and cost.

### SUMMARY

Some embodiments of the present disclosure relate to use of generative AI models to transform an image from first domain to second domain, leverage already developed technology or tools of second domain, and transfer results to first domain. A computer-implemented method includes accessing a first image from a first domain. The first domain may corresponds to one or more particular imaging modalities. The first domain may further corresponds to one or more particular stains if the first image is a digital pathology image.

The method may further include generating a virtual synthetic image by processing the first image using a machine learning model. The virtual synthetic image may belong to a second domain that corresponds to a different imaging modality or a different stain relative to the first domain. An image-processing tool may be accessed that is configured for processing images in the second domain. The image-processing tool may use segmentation, classification, or object detection models. Consequently, one or more annotations of the virtual synthetic image can be generated by processing the virtual synthetic image using the image-processing tool. The one or more annotations of the virtual synthetic image may be transferred to the first image. Moreover, an analysis using the first image and the transferred one or more annotations may be performed in the first domain.

According to some embodiments, the machine learning model may include a cycleGAN. In some instances, the cycleGAN may be trained on and is configured to generate virtual H&E based on the multiplex images. The first domain may include darkfield multiplex and the second domain may include H&E. The first domain may include immunohistochemistry and the second domain may include H&E. Additionally or alternatively, the first domain or the second domain may include magnetic resonance imaging or other radiology imaging. The one or more annotations may include an annotation of a tumor, stroma or artifact region. The one or more annotations may also include a segmentation of each of a set of cells.

Various techniques disclosed in the present disclosure can be utilized for leveraging existing tools or models in well-developed imaging domain to transfer them into other domains or new domains in which those tools either do not exist or it is difficult to develop them.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present disclosure is described in conjunction with the appended figures.

FIG. 1 shows an illustrative example of traditional multiplex image analysis that requires extra hematoxylin and eosin (H&E) slides from adjacent section to confirm the tumor and/or stroma segmentation.

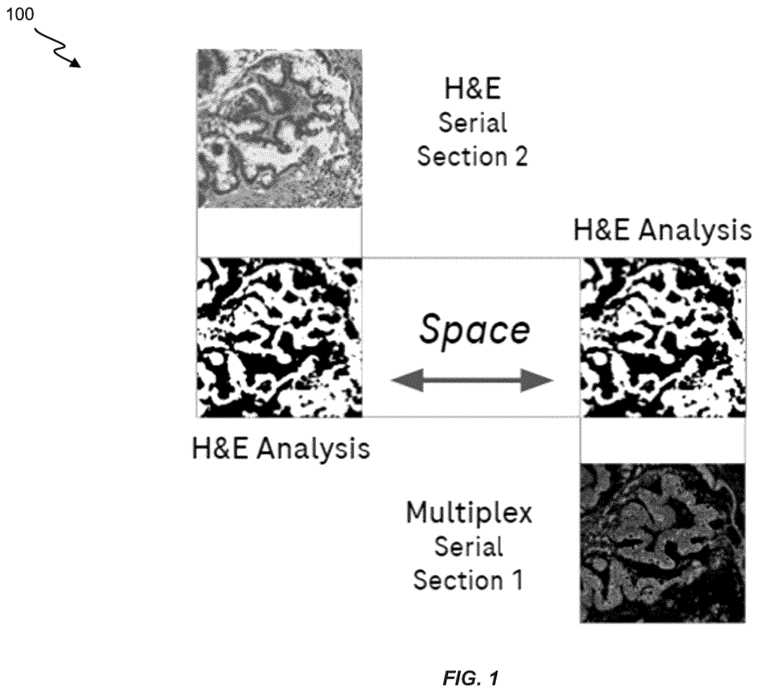

FIG. 2 is a block diagram illustrating an example overview of a system for performing domain swap by generating virtual synthetic images in accordance with an embodiment of the present disclosure.

FIG. 3 illustrates exemplary block diagram of performing a method for domain swap and subsequent processing using existing tools of a second domain in accordance with an embodiment of the present disclosure.

FIG. 4 shows an exemplary network of a digital pathology image generation system of FIG. 2.

FIG. 5 shows exemplary workflow of virtual staining by generating a synthetic virtual H&E image based on a multiplex image to bridge domain swap in accordance with an example implementation of the present disclosure.

FIG. 6 shows an illustrative example of a cycle generative adversarial network (CycleGAN) for generating the synthetic virtual H&E image from a multiplex digital pathology image, in accordance with an example implementation of the present disclosure.

FIG. 7 shows an illustrative example of biomarkers with different colors in individual channels of MPX fluorescence images.

FIG. 8 shows illustrative examples of tumor prediction and artifact detection based on the H&E images.

FIG. 9 shows an example illustration of tumor-stroma separation in real and synthetic H&E images in accordance with an example implementation of the present disclosure.

FIG. 10 illustrates an exemplary method of cells segmentation using the H&E image in accordance with some embodiments of the present disclosure.

FIG. 11 shows an example flowchart of a system performing domain swap and subsequent processing using existing tools of the second domain.

FIG. 12 illustrates a side-by-side comparison of the synthetic H&E image with the composite image or multiplex image and adjacent real H&E image in accordance with an example implementation of the present disclosure.

FIG. 13A illustrates a side-by-side comparison of the synthetic H&E image and segmented tumors in the synthetic H&E image in accordance with an example implementation of the present disclosure.

FIG. 13B shows an illustrative example of cells detected in the synthetic H&E image with segmented tumors and a zoomed in patch of cells detected in accordance with an example implementation of the present disclosure.

FIG. 14A shows an illustrative example of a multiplex image or the composite image with segmented tumor and cells, in accordance with an example implementation of the present disclosure.

FIG. 14B shows a zoomed in patch of the multiplex image of FIG. 14A with nuclei segmented results in dapi channel and in the composite image in accordance with an example implementation of the present disclosure.

### DETAILED DESCRIPTION

Some embodiments of the present disclosure relate to use of generative AI models to transform an image from a first domain to a second domain, leveraging previously developed technology or tools of the second domain, and transferring the results to the first domain for further processing of the image. The first domain or the second domain may correspond to different imaging modalities (e.g., radiology imaging, brightfield imaging etc.) or different stains in digital pathology (e.g. H&E staining, multiplex staining etc.). According to some embodiments, a technical solution is provided in the present disclosure to a technical problem of transferring tools and/or techniques to different imaging modalities.

The term “domain” (first domain or second domain) as used herein, may refer to a representation of an image using a specific stain or imaging modality. Exemplary domains include H&E staining of digital pathology slides, multiplex staining of digital pathology slides (with any combination of stains), singleplex staining of digital pathology slides (with any given stain), immunohistochemistry (using any given antibody). The exemplary domains may further include imaging modalities such as darkfield microscopy, brightfield microscopy, fluorescent microscopy, or radiology imaging (e.g., magnetic resonance imaging (MRI), computed tomography (CT), positron emission tomography (PET), etc.).

As used herein, the term “same space” refers to a space that has identical spatial reference points in the same original image. Different domains can exist or can be represented in the same space. For example, a first domain can include a singleplex domain that uses a first particular stain, and a second domain can include a different singleplex domain that uses a different particular stain. In another example, a section (tissue slice) may be stained with a first dye or stain and may be scanned to obtain a singleplex image in the first domain. Subsequently, the same exact section may be stained sequentially with another different dye or stain and can be scanned to obtain a duplex image in the second domain. Both of these images (singleplex and duplex) may belong to different domains but are represented in the same space. Similarly, two different stains or labeling in an exact same section such as imaging the same fluorescence immunohistochemistry sample sequentially for two fluorophores may produce images in the same space. Different images with different spatial reference points can represent the same domains (e.g., two H&E images in a set of serial or adjacent sections).

In some embodiments of the present disclosure, an image from the first domain is processed using a machine learning model (e.g., a cycleGAN model, a Pix2pix GAN, a generative pre-trained transformer model) to generate a virtual synthetic image in the second domain. This approach can eliminate or reduce the need to separately collect an image in the second domain, which can save time and cost. Tools that are available in the second domain can be used to perform image processing on the virtual synthetic image. For example, segmentation and/or categorization can be performed. Results from the image processing can then be easily and simply used to assess the image from the first domain, given that they have an identical size, scale, view, etc. Thus, pixels identifying a boundary of a region depicted in the virtual synthetic image are the same pixels in the image from the first domain. The disclosed technique can be used across a variety of domain combinations.

In the present disclosure, the term “singleplex” may refer to an image that displays a single staining component or a marker. The term is often used in contrast to a “multiplex” or “MPX” image, which involves the simultaneous visualization of multiple staining components within a single cell or tissue sample. The term “sample” may be understood as material derived from a biological organism, comprising but not limited to hair, skin samples, tissue samples, cultured cells, cultured cell media, and biological fluids. The term “tissue” refers to a mass of interconnected cells (e.g., central nervous system (CNS) tissue, liver tissue, or eye tissue) derived from a human or other animal. Samples may include the connecting material and the liquid in association with the cells, such as blood samples. In the context of histopathology, the term “slide” refers to a glass microscope slide carrying a thin section of tissue that has been stained for microscopic examination. The term sample may also include media containing isolated cells. One skilled in the art may determine the quantity of samples required to obtain a reaction by standard laboratory techniques. Additionally, the term adjacent slide or sequential slide refers to a slide that includes the next consecutive tissue slice from the same sample used to prepare an original slide. These adjacent slides may be used for cross validation or as reference for the original slide analysis, allowing researchers to interpret results. For instance, the original slide may be stained with a given set of dyes or markers (e.g., multiplex) and the next slide is stained with another set of dyes or markers (e.g., H&E).

The term “biomarker” as used herein refers to a characteristic of tissue including, but not limited to, the presence of a particular cell type such as immune cells, particularly those indicative of a medical condition. The identification of the biomarker may involve the presence of a particular molecule, such as a protein within the tissue feature.

The term “marker” is herein defined as a stain, dye, or tag utilized to distinguish a biomarker from surrounding tissue or other biomarkers. The tag, which may include an antibody—specifically one exhibiting a high affinity for a protein associated with a particular biomarker—can be employed for labeling purposes. A marker may demonstrate a high affinity for a specific biomarker, such as a particular molecule or protein associated with a disease. The biomarker to which a marker associates with may be distinct or exclusive to the respective marker. Furthermore, a dye-based marker may impart coloration to tissue, thereby indicating the presence of a biomarker within the tissue. Various stain and dye-based markers may appear with distinct colors in the sample allowing for multiple markers to be used in combination.

Similarly, in the context of immunohistochemistry (IHC), an IHC marker refers to an antibody specifically designed to bind to a target protein or antigen within tissue sections. IHC markers may conjugate to various tags (e.g., chromogens, quantum dots, or fluorophores) to facilitate visualizing and identifying specific cellular components or biomolecules within tissues. IHC markers thereby aid in the characterization and diagnosis of various medical conditions or research purposes.

Differential staining is fundamental to pathology and encompasses the staining of markers associated with cytoplasm, organelles including the nuclei, and specific proteins. A prime illustration may be hematoxylin-eosin (H&E) staining, where hematoxylin (blue) predominantly stains cell nuclei, while eosin (magenta-red) serves as a cytoplasmic stain. Differential staining can increase contrast in the sample and allow for the easy identification of cellular components. The ratio of hematoxylin and eosin staining in the cytoplasm may also provide insights into its basophilic or acidophilic characteristics. Another common application of differential staining involves IHC staining, which can highlight the presence of specific epitopes based on antigen-antibody binding. In IHC staining a unique, high-specificity antibody can be developed for almost any target (or biomarker), which can also be conjugated with various tags. As it may be very difficult to find a stain or dye to label any random two or three target proteins with different colors, therefore, IHC staining is commonly used for differential staining and/or multiplex staining. Pathologists frequently utilize IHC techniques for cancer diagnostics, to identify immune cells, assess the expression of tumor and cell proliferation markers, and to detect conditions such as degenerative disorders and infectious diseases.

In immunohistochemistry, the term “labeling” refers to adding a tag or marker to an antigen to aid in its detection. There are two main types of IHC labeling, fluorophoric and chromogenic. Fluorophoric uses compounds, called fluorophores, that produce a fluorescent signal when excited by light (e.g., ultraviolet (UV) or visible light). Recently, Quantum dot (QD) labeling has attracted a lot of attention. Quantum dots (QDs) are semiconductor nanocrystal fluorophores with extremely high fluorescence efficiency and low photobleaching. Due to their quantum effect and size effect, QDs possess a constant excitation wavelength together with sharp and symmetrical tunable emission spectra. A fluorescent microscopy can be used to view the fluorophoric labeled slide or a specimen. Fluorescence imaging can be performed typically with a monochrome camera combined with multiple filter sets that match the absorbance and emission characteristics of each fluorophore. In addition, a darkfield microscopy is a technique that utilizes oblique illumination to enhance contrast in specimens that are not imaged well under normal illumination conditions. For example, cell structures that may appear transparent with brightfield illumination can be viewed with better contrast and detail using darkfield. Chromogenic labeling, on the other hand, relies on an enzyme/substrate reaction to produce a pigmented deposit, and can be observed using a brightfield microscopy. Some chromogens include but are not limited to Di-Amino-Benzidine (DAB), Amino-Ethyl-Carbazole (AEC), Bajoran Purple™, Vina Green™, Fast Red (FR).

Immunohistochemistry technique can be used to study multiple biomarkers or antigens in the same tissue section. The IHC technique may provide comprehensive information about different cellular interactions, tissue heterogeneity, antigen localization and co-localization, functional states, distribution of antigens, and relative concentration. In addition, multiplex staining saves cost, time, and effort to prepare multiple slides for each stain or biomarker and allows for the joint or relative analysis of different cell populations on the same tissue section. Multiplex IHC staining involves multiple primary antibodies, each recognizing a specific target. Afterwards, corresponding secondary antibodies may also be applied to enhance signal amplification as more than one secondary antibody molecule can bind to each primary antibody. The chromogens or fluorophores can be either coupled with the primary antibody (the direct method) or with the secondary antibody (the indirect method) for labeling antigens.

When a fluorophore is used to visualize an IHC target, the technique may be referred to as fluorescent immunohistochemistry (f IHC) or immunofluorescence staining (IF). Multiplex fluorescent immunohistochemistry (mf IHC) may be used to label multiple targets in the same sample by conjugating either the primary or secondary antibodies with fluorophores with different absorption and emission spectra. The different fluorophores may be imaged simultaneously or sequentially, for example, by using fluorescence imaging. Each fluorophore may correspond to a specific channel that represents the location of the targeted antigen or biomarker. The channels may then be combined into a single composite image or viewed separately.

One exemplary embodiment of the disclosure relates to generating a synthetic reference image (e.g., a synthetic virtual H&E staining image) by processing a multiplex image using a machine learning model (e.g., a cycleGAN model, a Pix2pix GAN model, or a generative pre-trained transformer model). The multiplex image is transferred from a multiplex domain (in which multiple biomarkers are stained) to a virtual H&E domain. This approach can eliminate the need to stain, image, and process sections with a reference stain, which can result in saving resources. Further, processing tools that have been developed in the H&E domain can then be used to analyze the virtual synthetic reference image (e.g., to segment tumors and/or stroma regions, to detect artifacts, segment cells, etc.). Boundaries and/or areas (e.g., of a tumor region, stroma region, artifact depiction, and/or one or more cells) can then be easily mapped to the multiplex domain and the multiplex image, since the reference points are the same.

Another exemplary embodiment relates to using synthetically generated images that can be used for validation purposes for model execution and/or for fine-tuning virtual-image results. For instance, if algorithms or tools are developed in IHC domain, then the virtual staining H&E images can be generated using IHC images. The tools available in the IHC domain may then be transferred and applied directly to H&E domain. Thus, it will be appreciated that tools and algorithms developed in one domain can be transferred using the generated virtual slides from the other domains.

Various techniques disclosed in the present disclosure can be utilized for leveraging existing tools or models in well-developed imaging domains to transfer them into other domains or new domains in which those tools either do not exist or it is difficult to develop them.

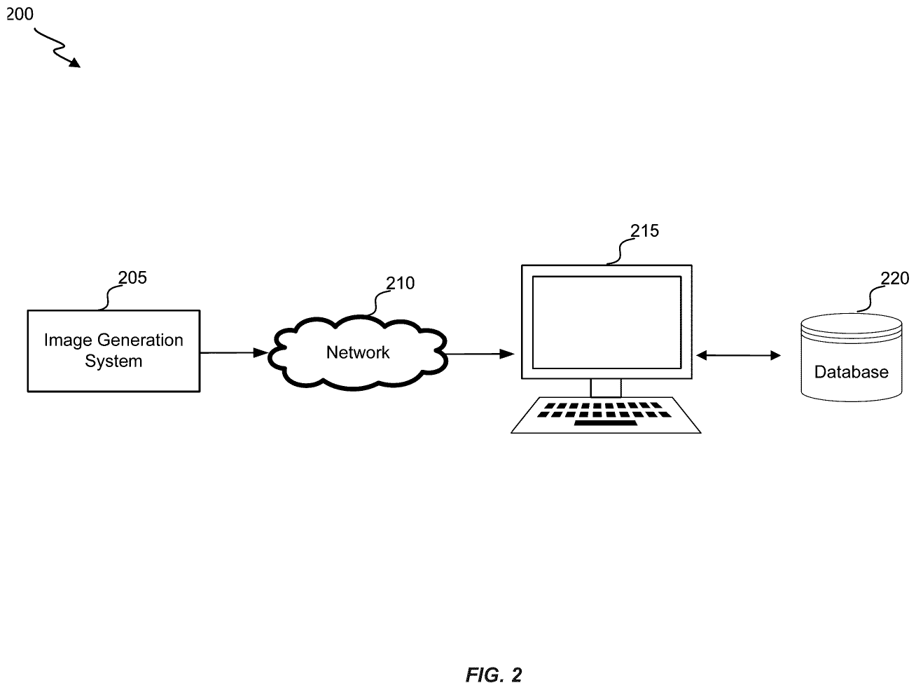

FIG. 2 is a block diagram illustrating an example overview of a system for performing domain swap by generating virtual synthetic images in accordance with an embodiment of the present disclosure. Exemplary system 200 may include an image generation system 205 connected to one or more computer systems 215 through a network 210. The described technique of domain swapping, which creates virtual synthetic images in the second domain by applying generative AI models to images from the first domain, can be executed on computer system 215. The computer system 215 may also include user input and output devices (not shown) such as a keyboard, mouse, stylus, and a display/touchscreen. The computer system 215 may receive one or more images of first domain from the image generation system 205 and generate images of second domain. In addition, the computer system 215 may be used to execute image processing tools of the second domain and to store the results or images in one or more databases 220.

The computer system 215 of the exemplary system 200 may include a processing system with one or more processors, high-speed central processing unit(s) (CPU), and one or more memories. The computer system 215 may also include a memory for storing a plurality of processing modules or logical instructions that are executed by the one or more processors coupled. The computer memory that stores data may also be maintained on a computer readable medium including magnetic disks, optical disks, organic memory, and any other volatile (e.g., random access memory (RAM)) or non-volatile (e.g., read-only memory (ROM), flash memory, etc.) mass storage system readable by the CPU. The computer readable medium may include cooperating or interconnected computer readable medium, which exist exclusively on the processing system or can be distributed among multiple interconnected processing systems that may be local or remote to the processing system.

The network 210 may include, internet, an intranet, a wired LAN (local area network), a wireless LAN (WiLAN), a WAN (wide area network), a MAN (metropolitan area network), a PSTN (public switched telephone network) and other types of communications networks. The network 210 may further include communication devices such as one or more gateways, routers, or bridges. Merely by way of example, the network 210 can have one or more servers and one or more web-sites accessible by users to send and receive information usable by the computer system 215. The network 210 may be any type of network familiar to those skilled in the art that can support data communications using any of a variety of available protocols, including without limitation TCP/IP (transmission control protocol/Internet protocol), SNA (systems network architecture), IPX (internet packet exchange), AppleTalk®, and the like.

The exemplary system 200 may further include the one or more databases 220 for the processing and storing of data (e.g., histopathology images). The one or more databases 220 may be integral to a memory system on the computer or in secondary storage such as a hard disk, floppy disk, optical disk, or other non-volatile mass storage devices. The computer system 215 may include a client terminal in communication with one or more servers, or personal digital/data assistants (PDA), laptop computers, mobile computers, internet appliances, one or two-way pagers, mobile phones, or other similar desktop, mobile or hand-held electronic devices.

For instance, the computer system 215 may provide a means for inputting image data depicting one or more scanned digital pathology slides from the image generation system 205 to memory. The image data may include data related to color channels (RGB) for brightfield imaging. In fluorescence imaging the image data may include data related to multiple distinct channels. Each channel may correspond to or be responsible for capturing a particular spectral range or (signal) wavelengths emitted from the fluorophores. Hence, each channel provides image with representation of a specific biomarker. For instance, a biological specimen, for example, a tissue section may need to be stained by means of application of a staining assay to highlight one or more different biomarkers associated with chromogenic stains for brightfield imaging or fluorophores for fluorescence imaging. Staining assays can use chromogenic stains for brightfield imaging, organic fluorophores, quantum dots, or organic fluorophores together with quantum dots for fluorescence imaging, or any other combination of stains and viewing or imaging devices. In the analysis of biological specimens, for example, cancerous tissues, different stains are specified to identify one or more types of biomarkers, for example, immune cells.

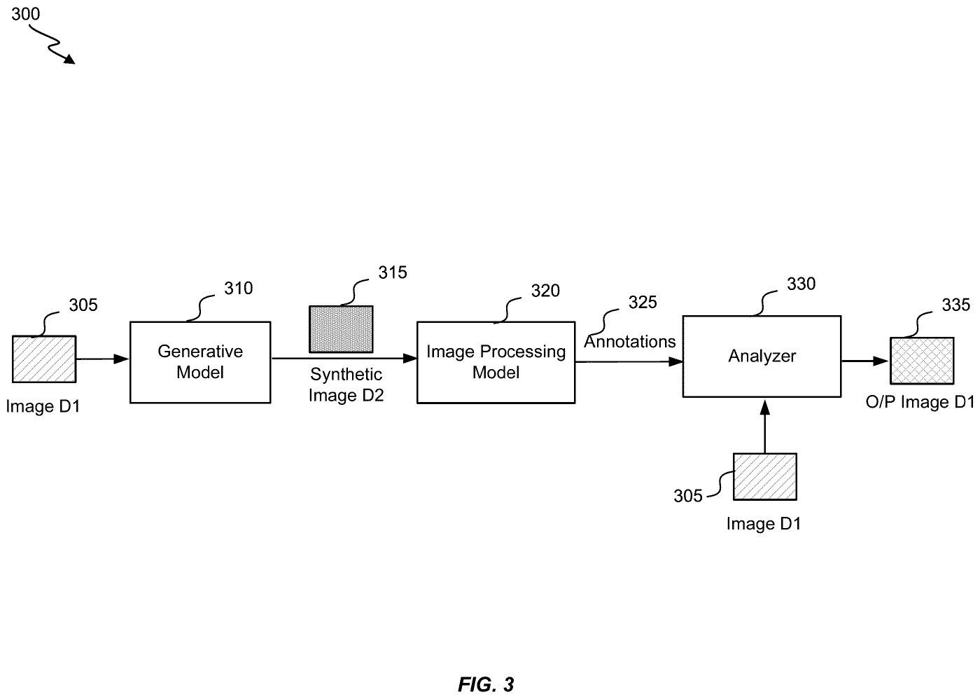

FIG. 3 illustrates an exemplary block diagram of performing a method for domain swap and subsequent processing using existing tools of the second domain. In some embodiments, an image D1 305 from the first domain is processed using a generative model 310 (e.g., a cycleGAN model, a Pix2pix GAN, a generative pre-trained transformer model) to generate a synthetic image D2 315 in the second domain. For example, the image D1 305 from the first domain can be a multiplex IHC image and the synthetic image D2 315 in the second domain may represent the H&E. This approach can eliminate or reduce the need to separately collect an image in the second domain but still allow the user to employ tools developed for use in the second domain. This approach can save time and cost. The synthetic image D2 315 may be processed by an image processing model 320. The image processing model 320 may include models or tools that are available in the second domain and can be used to perform image analysis of the synthetic image D2 315. For example, segmentation and/or categorization can be performed by the image processing model 320 to generate annotations 325. The results or annotations 325 from the image processing model 320 can be mapped on the image D1 305 by an analyzer 330. The mapping can be performed easily because the image D1 305 and the synthetic image D2 315 have an identical size, scale, and view, etc. Thus, pixels identifying a boundary of a region depicted in the synthetic image D2 315 are the same pixels in the image D1 305 of the first domain. After mapping the annotations 325 on to the image D1 305, the analyzer 330 may be used to perform additional analysis on the image D1 305 based on the annotations 325 and to generate an output image D1 335 of the first domain.

According to some aspects of the present disclosure, the disclosed technique can be used across a variety of domain combinations. The domain (e.g., first domain or second domain) as used herein refers to a representation of an image using a specific stain or imaging modality. Exemplary domains include but are not limited to H&E staining, multiplex staining (with any combination of stains), singleplex staining of digital pathology slides (with any given stain), and labeling via immunohistochemistry (using any given antibody). Exemplary domains further include imaging modalities such as darkfield microscopy, brightfield microscopy, fluorescence microscopy, or even radiology scans such as MRI, CT, X-rays, PET, etc. Different domains can exist or can be represented in the same space. The image D1 305 of first domain and the synthetic image D2 315 of the second domain, both will represent identical spatial reference points and can be considered in the same space. For example, the first domain can include an image with multiplex staining, and the second domain can include an image with H&E staining.

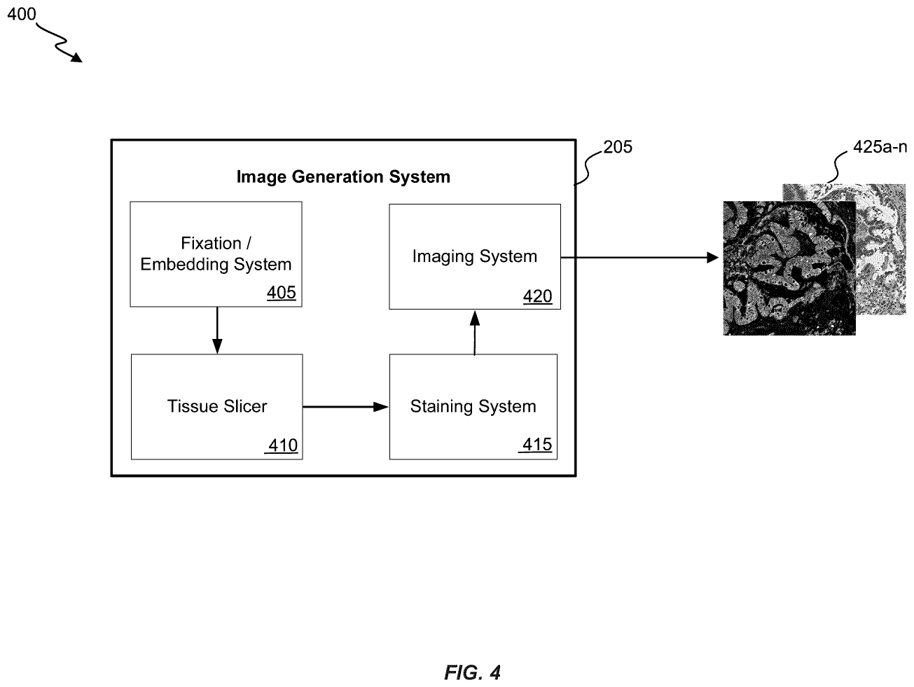

FIG. 4 illustrates an exemplary network of the image generation system 205 of FIG. 2 to generate digital pathology images. The image generation system 205 may include a fixation/embedding system 405, a tissue slicer 410, a staining system 415, and an imaging system 420. The fixation/embedding system 405 fixes and/or embeds a tissue sample (e.g., a liquid fixing agent, such as formaldehyde solution) and/or an embedding substance (e.g., a historical wax, such as paraffin wax and/or one or more resins, such as styrene or polyethylene). Each slice may be fixed by exposing the slice to a fixating agent for a predefined period of time (e.g., at least 3 hours) and by then dehydrating the slice (e.g., via exposure to an ethanol solution and/or a clearing intermediate agent). The embedding substance can infiltrate the slice when it is in liquid state (e.g., when heated).

The tissue slicer 410 then slices the fixed and/or embedded tissue sample (e.g., a sample of a tumor) to obtain a series of sections, with each section having a thickness of, for example, 4-5 microns. Such sectioning can be performed by first chilling the sample and then slicing the sample in a warm water bath. The tissue can be sliced using (for example) a vibratome or compresstome.

Because the tissue sections and the cells within them are virtually transparent, preparation of the slides typically includes staining (e.g., automatically staining) the tissue sections to render relevant structures more visible. In some instances, the staining is performed manually. In some instances, the staining is performed semi-automatically or automatically using the staining system 415.

The staining can include exposing an individual section of the tissue to one or more different stains (e.g., consecutively, or concurrently) to express different characteristics of the tissue. For example, each section may be exposed to a predefined volume of a staining agent for a predefined period of time. The staining agent can include (for example) an RNA probe, protein probe (e.g., nuclear-protein probe or cytoplasm-protein probe), an immunohistochemistry stain, a probe for a secreted substance, etc. In some instances, the staining agent is one that stains for KAPPA mRNA or LAMBDA mRNA.

One exemplary type of tissue staining is histochemical staining, which uses one or more chemical dyes (e.g., acidic dyes, basic dyes) to stain tissue structures. Histochemical staining may be used to indicate general aspects of tissue morphology and/or cell microanatomy (e.g., to distinguish cell nuclei from cytoplasm, to indicate lipid droplets, etc.). One example of a histochemical stain is hematoxylin and eosin (H&E). Other examples of histochemical stains include trichrome stains (e.g., Masson's Trichrome), Periodic Acid-Schiff (PAS), silver stains, and iron stains. The molecular weight of a histochemical staining reagent (e.g., dye) is typically about 500 kilodaltons (kD) or less, although some histochemical staining reagents (e.g., Alcian Blue, phosphomolybdic acid (PMA)) may have molecular weights of up to two or three thousand kD. One case of a high-molecular-weight histochemical staining reagent is alpha-amylase (about 55 kD), which may be used to indicate glycogen.

Another type of tissue staining is immunohistochemistry (IHC, also called “immunostaining”), which uses a primary antibody that binds specifically to the target antigen of interest (biomarker). IHC may be direct or indirect. In direct IHC, the primary antibody is directly conjugated to a label (e.g., a chromophore or fluorophore). In indirect IHC, the primary antibody is first bound to the target antigen, and then a secondary antibody that is conjugated with a label (e.g., a chromophore or fluorophore) is bound to the primary antibody. The molecular weights of IHC reagents are much higher than those of histochemical staining reagents, as the antibodies have molecular weights of about 150 kD or more.

The sections may then be individually mounted on corresponding slides. The imaging system 420 can then scan the slides to generate digital-pathology images 425a-n. Each section may be mounted on a slide, which is then scanned to create a digital image that may be subsequently examined by digital pathology image analysis and/or interpreted by a human pathologist (e.g., using image viewer software). The imaging system 420 may digitize pathology slides (whole slide or a section) using bright-field imaging, dark-field imaging, or fluorescence imaging. The imaging system 420 can include but is not limited to microscope with digital camera, robotic microscopes, or WSI scanners such as Ventana iScan HT, Ventana DP 200, or Ventana DP 600.

In some instances, a pathologist may review and manually annotate the digital image of the slides (e.g., tumor area, necrosis, etc.). Annotation of regions of interest may be performed automatically using a computer-vision technique. Digital-pathology images 425a-n may be converted into other domains for further processing.

A digital histopathology image (e.g., 425a) typically includes an array, usually a rectangular matrix, of pixels. Each “pixel” is one picture element and is a digital quantity that represents some property of the image at a location in the array corresponding to a particular location in the image. Typically, in continuous tone black and white images the pixel values represent a gray scale value. Pixel values for a digital image typically conform to a specified range. For example, each array element may be one byte (i.e., eight bits) representing pixel values in the range of 0 to 255. In a gray scale image, a “255” may represent absolute white and zero (‘0’) an absolute black (or visa-versa). Color images may comprise of three-color planes, generally corresponding to red, green, and blue (RGB). For a particular pixel, there is one value for each of these color planes, (i.e., a value representing the red component, a value representing the green component, and a value representing the blue component). By varying the intensity of these three components, all colors in the color spectrum are typically created. A specimen stained by multiplex IHC may be illuminated sequentially with multiple light channels matched to the absorbance bands of the chromogens to capture brightfield images. In the case of multiplex immunofluorescence, fluorescence microscopy with different filters may be used to capture fluorescence or emitted light from fluorophores associated with each biomarker.

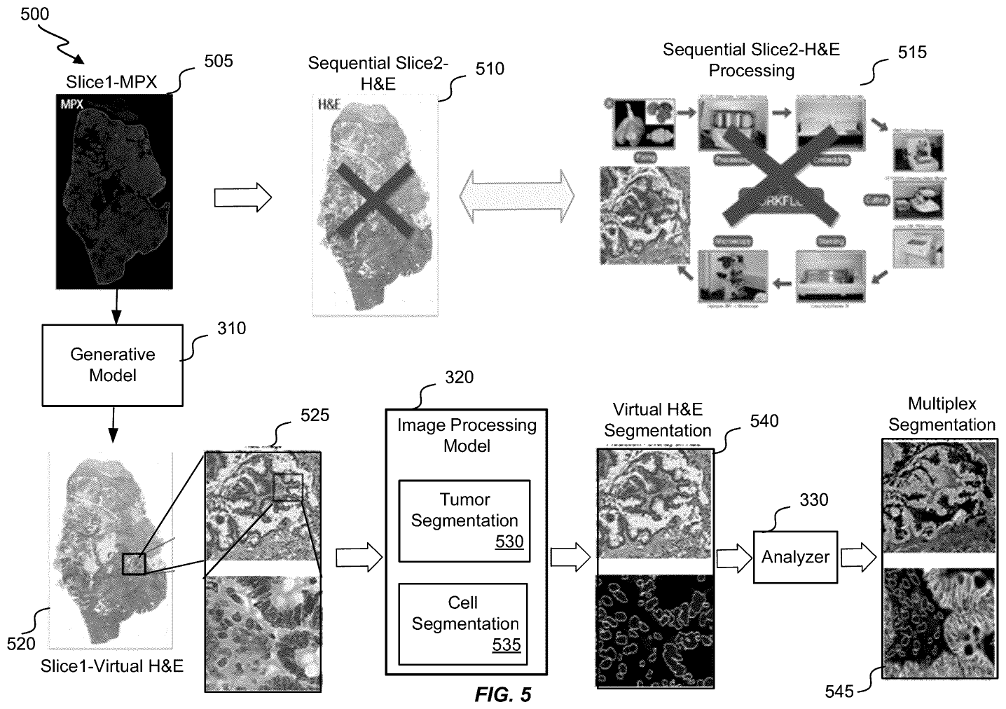

FIG. 5 shows an exemplary workflow of virtual staining by generating a synthetic H&E image based on a multiplex image and to a bridge domain swap. Traditionally, sequential tissue slices from the sample are stained with different dyes or techniques for disease specific analysis. For example, a multiplex IHC staining may be performed on a tissue slice resulting in an image as shown as slice1-MPX 505 in FIG. 5. As H&E stains provide better morphological information, a sequential slice of the sample can be stained with H&E as depicted by sequential slice 2-H&E 510. This staining of sequential slice 2-H&E 510 requires additional processing such as obtaining sequential tissue slice, preparation of the glass slide, staining, or scanning etc. and leads to additional time, cost, and labor. Moreover, due to the typical thickness of tissue slices (3-4 microns) as compared to a typical cell's size (4-20 microns), a given cell might not be visible on two sequential slices of sample. Consequently, the spatial reference points alignment between the two images (e.g., slice1-MPX 505 and sequential slice 2-H&E 510) would be difficult and artifacts on either slide or image can ruin the analysis.

In some embodiments of the present disclosure, multiplex images can be employed to generate highly realistic corresponding virtual H&E images. When these virtual H&E images are analyzed using algorithms designed for H&E, they yield results that closely mirror those results obtained from analysis of actual H&E images, achieving a high level of accuracy. For example, the generative model 310 can be used to create slice1-virtual H&E 520 image based on the slice1-MPX 505 image. The image processing model 320 may include tumor segmentation 530 or cell segmentation 535 may then be utilized on slice1-virtual H&E 520 image or its patch 525 to generate virtual H&E segmentation 540. The analyzer 330 may utilize the virtual H&E segmentation 540 to produce multiplex segmentation by mapping the tumor or cells on the slice1-MPX 505, as indicated by an image patch 545 of multiplex image. This mapping by the analyzer 330 is possible as the spatial reference points are the same between slice1-virtual H&E 520 (second domain) and slice1-MPX 505 (first domain). Thus, according to disclosed virtual staining and domain swap technique, annotations and other data collected in one domain can be transferred seamlessly to the other domain.

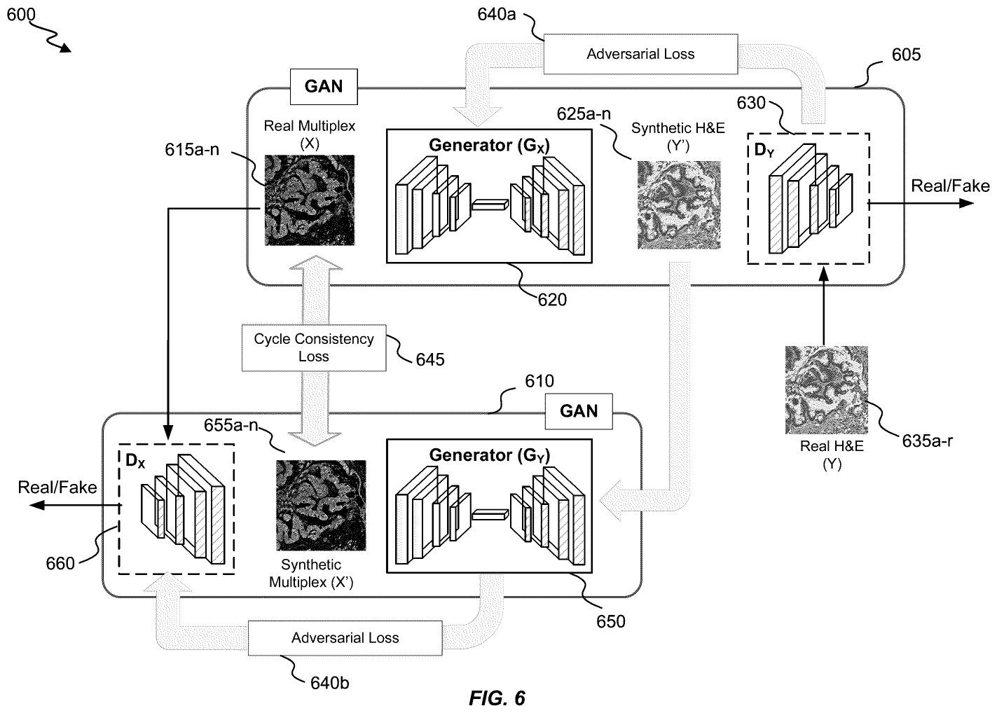

FIG. 6 shows an illustrative example of a cycle generative adversarial network (CycleGAN) 600 for generating the synthetic virtual H&E image from a MPX digital pathology image, in accordance with an example implementation of the present disclosure. FIG. 6 illustrates how a CycleGAN model is used as a deep-learning approach to transform an image from a first domain to a second domain. In the depicted instance, the first domain is a 6-channel multiplex domain, and the second domain is an H&E domain. The 6-channel multiplex domain may be represented by a 6-channel multiplex image also referred hereinafter as a composite image or simply multiplex image. The multiplex image may emphasize six different stains or biomarkers. In some cases, 6-channel multiplex domain can also be represented by MPX fluorescence images or MPX darkfield images associated with individual channels (e.g., six channels fluorescence—MPX darkfield images). Each image of the darkfield images may correspond to a different channel and may depict a different biomarker.

CycleGAN 600 is a type of GAN that is specifically designed for unpaired image-to-image translation. It is commonly used for tasks such as style transfer, image colorization, and image transformation. The key innovation of CycleGAN 600 is its ability to learn mappings between two domains (e.g., a multiplex image to a H&E image, horses to zebra, or a noisy to a denoised) without requiring paired data samples from both domains during training. In CycleGAN 600, there are two GANs 605 and 610 one for each domain. Each GAN in the CycleGAN 600 may further comprise of two main components: a generator network (e.g., 620 and 650) and a discriminator network (e.g., 630 and 660), similar to other GAN architectures. For example, to translate images from domain X or first domain (multiplex) to domain Y or second domain (H&E) and vice versa, a generator G_{X} 620 network with mapping X→Y and inverse mapping generator G_{Y} 650 for Y→X may be dedicated, respectively.

Each generator may take one or more images from its respective domain, for example, the generator G_{X} 620 may take a real multiplex image (X) 615a as input and output a transformed image, e.g., a synthetic H&E image (Y′) 625a. This transformed image may resemble the target domain i.e., real H&E images (Y) 635a-r e.g., a dataset of hematoxylin or eosin stains images. In CycleGAN 600, there are also two discriminators, one for each domain, denoted as D_{Y} 630 and D_{X} 660. These discriminators aim to distinguish between real images from the target domain and fake or synthetic images produced by the generators. For example, the discriminator D_{Y} 630 may aim to distinguish between the real H&E images (Y) 635a-r and the synthetic H&E images (Y′) 625a-n from the generator G_{X} 620. Similarly, the discriminator D_{X} 660 may be trained to distinguish between the real multiplex images (X) 615a-n and fake or synthetic multiplex images (X′) 655a-n from the generator G_{Y} 650. In each GAN (605 and 610), the generator and discriminator are trained in an adversarial manner, which involves a competitive process between the two networks.

In CycleGAN 600, the generators and discriminators facilitate the translation of images between two domains while preserving the semantic content. The generators may employ a deep neural network architecture such as convolutional neural network (CNN), transformer-based architectures, or residual network (ResNet) that may leverage multiple layers to extract and transform features at different abstraction levels. For example, the generator may comprise of encoder decoder components, where the encoder extracts high level features from the input image and decoder reconstructs these features into the targeted domain. On the other hand, discriminators are binary classifiers that assess the authenticity of an image to be real or fake. Both the discriminators may have a different or similar architecture utilizing neural networks such as CNN to analyze and compare features of the synthetic images with those of real images in the respective domains. It may be appreciated that the network architecture of both the generators and both the discriminators can be a variant of a neural network. Both the generators may have a similar or a different network architecture, being trained in an adversarial manner while maintaining cycle consistency.

The goal or focus of the generator is to produce synthetic images that are indistinguishable from real images. While the goal or focus of the discriminator is to correctly classify real images as real and synthetic images as fake. The adversarial objective or loss (e.g., 640a and 640b) is one of the primary components of a GAN and is responsible for training the generators to produce realistic-looking images. The adversarial loss 640a for training the generator G_{X} 620 and the discriminator D_{Y} 630 to transform the real multiplex images (X) 615a-n to the synthetic H&E images (Y′) 625a-n may be formulated as:

$${Loss}_{adv} ( {G}_{X} , {D}_{Y} , X ) = \frac{1}{n} {\sum }_{i = 1}^{n} {( 1 - {D}_{Y} ( {G}_{X} ( {x}_{i} ) ) )}^{2}$$

where n is the total number of samples xᵢ in the training set X of real multiplex images 615a-n.

For inverse mapping, the adversarial loss 640b for training the generator G_{Y} 650 and the discriminator D_{X} 660 to translate the synthetic H&E images (Y′) 625a-n to the synthetic multiplex images (X′) 655a-n may be formulated as:

$${Loss}_{adv} ( {G}_{Y} , {D}_{X} , Y ) = \frac{1}{n} {\sum }_{i = 1}^{n} {( 1 - {D}_{X} ( {G}_{Y} ( {y}_{i}^{′} ) ) )}^{2}$$

where y′ᵢ represents a sample synthetic image from the set Y′ of synthetic H&E images 625a-n.

The principle of CycleGAN 600 is to translate the image from one domain to the other and back as a cycle. Hence, a cycle consistency loss (Loss_{cyc}) 645 between an original input (real multiplex image 615a-n) and a final synthetic image (synthetic multiplex image 655a-n) can be calculated with the goal to achieve consistency across both domains. Cycle consistency loss 645 may ensure that when an input image from domain X is translated to domain Y and then back to domain X, it resembles to a high degree with the input image from domain X. Cycle consistency loss 645 may help to maintain the mapping between different domains and prevent information loss during translation. The cycle consistency loss may achieve that G_{Y}(G_{X}(x))≈x (i.e., the generator G_{Y} may produce synthetic image (x′) based on synthetic image (y′=G_{X}(x)), that may be highly similar to original image x) and G_{X} (G_{Y} (y))≈y, as:

$${Loss}_{cyc} ( {G}_{X} , {G}_{Y} , {D}_{X} , {D}_{Y} , Y ) = \frac{1}{n} {\sum }_{i = 1}^{n} {‖ {G}_{Y} ( {G}_{X} ( {x}_{i} ) ) - {x}_{i} ‖}_{1} + {‖ {G}_{X} ( {G}_{Y} ( {y}_{i}^{′} ) ) - {y}_{i}^{′} ‖}_{1}$$

where ∥.∥₁ denotes L1 loss or mean absolute error (MAE).

In CycleGAN 600, both the generators may also be enforced to preserve the color composition between the respective domains. To pursue this, an identity loss may be calculated by feeding an image from the respective domain through both the generator and its inverse (i.e., the generator from the opposite domain) and then computing the differences between the original inputs (i.e., xᵢ, yᵢ) and the reconstructed images (since G_{Y}(yᵢ)=x′ᵢ, (G_{X}(xᵢ)=y′ᵢ).), as given in the equation below. Identity loss may operate within the same domain and focus on maintaining the identity of individual images. While cycle consistency loss 645 may operate across different domains and focus on maintaining the consistency of mappings between domains.

$${Loss}_{iden} ( {G}_{X} , {G}_{Y} ) = \frac{1}{n} {\sum }_{i = 1}^{n} {‖ {G}_{Y} ( {y}_{i} ) - {x}_{i} ‖}_{1} + {‖ {G}_{X} ( {x}_{i} ) - {y}_{i} ‖}_{1}$$

Finally, the objective function can be formed by summing all loss terms and weighted by hyperparameters α, β, and γ as:

$${Loss}_{Total} = α [ {Loss}_{adv} ( {G}_{X} , {D}_{Y} , X ) + {Loss}_{adv} ( {G}_{Y} , {D}_{X} , Y ) ] + β {Loss}_{cyc} + γ {Loss}_{iden}$$

In some other embodiments, other deep learning models can also be used to perform domain swap. For example, contrastive unpaired image-to-image translation (CUT) models may also be used. The unpaired image-to-image translation may be based on patch-wise contrastive learning and adversarial learning. Compared to CycleGAN, CUT may learn to perform more powerful distribution matching. Moreover, FastCUT technique, a variant of CUT may be utilized as an alternative to CycleGAN for a lighter (requires less memory), and faster training. In some instances, pix2pix GAN may be used when the paired image dataset of domain 1 and domain 2 is available.

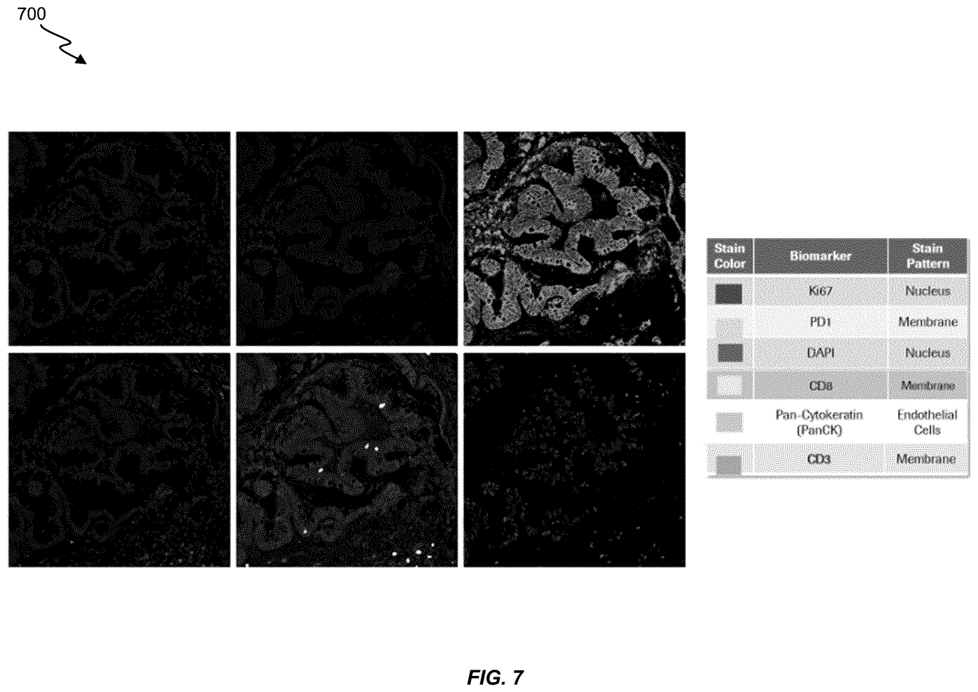

FIG. 7 shows an illustrative example of biomarkers with different colors in individual channels of MPX fluorescence images. Fluorescence imaging may be performed with a monochrome camera combined with multiple filter sets that match the absorbance and emission characteristics of each fluorophore. This allows imaging and isolation of as many fluorophores as spectral separation permits with the disadvantages that time is required to change between filter sets and separately collect and process different filtered images. The different filtered images or MPX fluorescence images (e.g., six images as shown in FIG. 7) can be combined to generate a composite image or a multiplex image (e.g., 6-channel multiplex image). The composite image is generated by integrating the pseudo colors from the different six channels of MPX, red for Ki67, cyan for PD1, blue color for DAPI, yellow for CD8, green for panCK, pink for CD3.

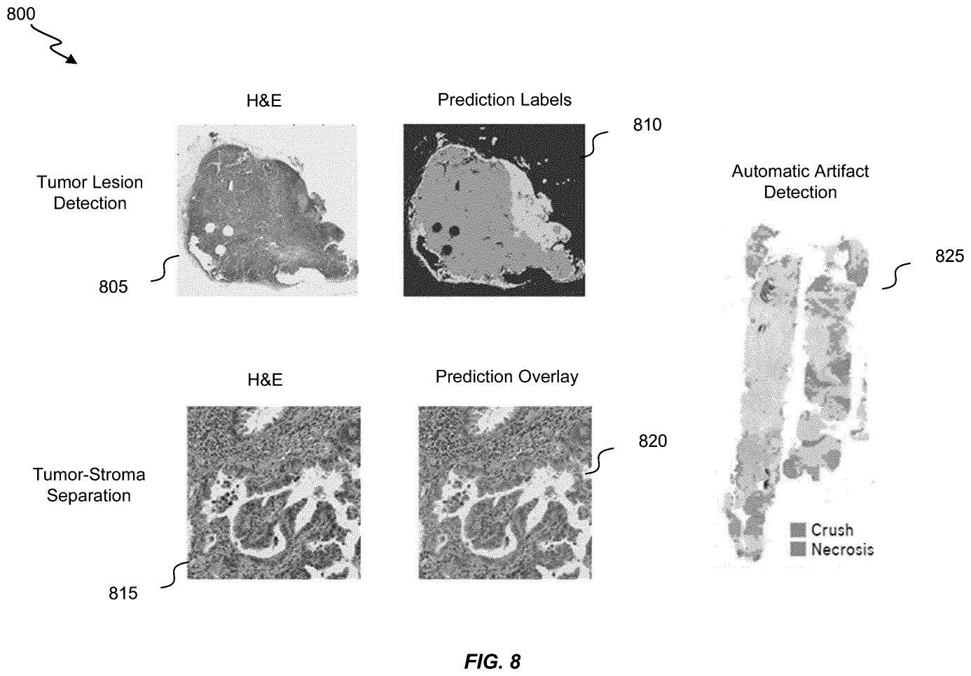

FIG. 8 shows illustrative examples of tumor prediction and artifact detection based on the H&E images. In digital pathology, machine learning models are often trained on and configured for H&E images for a variety of clinical use cases, which are mainly used to support clinical diagnostics and prognosis. For example, tumor lesion detection algorithm may be used to identify and delineate regions within the tissue that correspond to tumor lesions (or cancerous growths). In some instances, the tumor lesion detection algorithm can be a deep learning-based model such as convolutional neural networks (CNNs), which may be trained to recognize tumor-specific patterns, such as irregular cell shapes, increased cell density, and nuclear atypia. In other instances, approaches based on intensity-based thresholding or machine learning techniques with domain specific extracted features such as texture, shape, and intensity features from the H&E image patches can be used to discriminate tumors. As an example, the performance of tumor lesion detection algorithm on a H&E stained WSI 805 is illustrated in prediction labels image 810.

Further, tumor-stroma separation algorithm can also be used to distinguish between tumor epithelium (cancer cells) and stroma (surrounding connective tissue) within the same H&E image. Tumor-stroma separation algorithm can be based on deep learning techniques such as transfer learning by leveraging features learned by pre-trained CNNs to classify epithelial and stroma regions. In some other instances, machine learning models with extracted features (e.g., texture, color, shape, spatial cells arrangement related) may be used to segment tumor-stroma regions. Moreover, tumor-stroma ratio (TSR) can also be computed afterwards, which is a prognostic factor for survival in various types of cancers. For illustrative purposes, a prediction overlay image patch 820 is generated after processing a patch of H&E image 815 via tumor-stroma separation algorithm is shown in FIG. 8.

Similarly, in digital pathology, deep learning-based models can be trained and configured to remove artifacts from the digitized slides (or whole slide images—WSI). The artifacts in digital pathology images can obscure critical tissue regions, impacting diagnostic accuracy. Common types of artifacts include out-of-focus areas, tissue folds, ink marks, dust particles, pen marks, or air bubbles. Other forms of artifacts may include but are not limited to necrosis and crush etc. Necrosis may represent broader categories of cell death and can be resulted from various factors, including ischemia, physical agents, chemical agents, or immunological injury. Whereas a crush involves mechanical compression and tissue distortion. As an example, automatic artifact detection 825 of FIG. 8, highlights the crush and the necrosis regions in the H&E image.

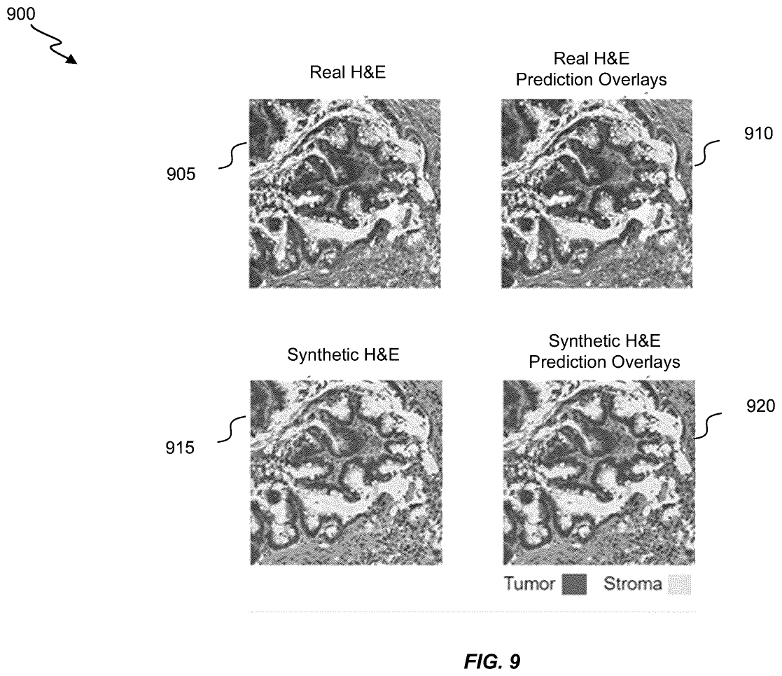

FIG. 9 shows an example illustration of tumor-stroma separation in real and synthetic H&E images in accordance with an example implementation of the present disclosure. A comparison of the performance of the tumor-stroma separation algorithm shows that the results (real H&E prediction overlays 910) from the application of the algorithm on a real H&E image 905 are to a large degree conserved in the results (synthetic H&E prediction overlays 920) of the same algorithm applied to a synthetic H&E image 915. This illustrative example demonstrates that H&E-domain tools configured to segment tumor and/or stroma regions and/or to detect artifacts can also be applied to synthetic virtually stained images with very similar results.

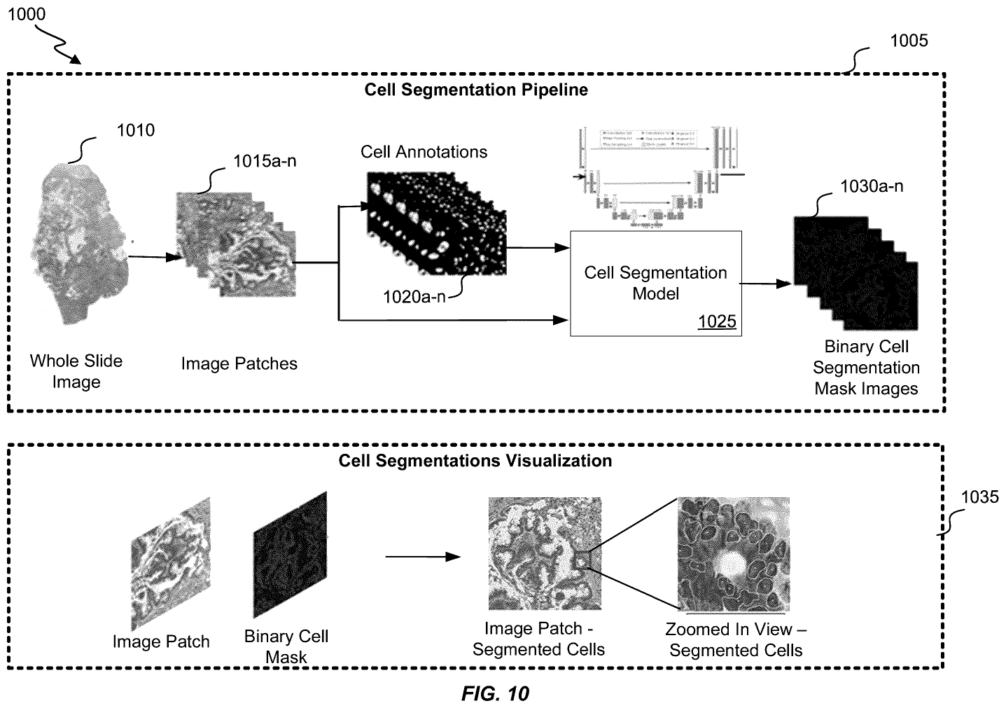

FIG. 10 illustrates an exemplary method of cells segmentation using the H&E image in accordance with some embodiments of the present disclosure. A first machine-learning model (e.g., a Cycle-GAN) may be used to transform an image in the first domain into the second domain, where the second domain is an H&E domain. A tool trained in the second domain can perform cell segmentation using a process as illustrated in FIG. 10. The boundaries of the cells are then mapped into the first domain and used for assessments, such as cell counting, tumor cellularity calculations and/or biomarker identifications.

A cell segmentation pipeline 1005 may include converting a whole slide image 1010 into image patches 1015a-n and obtaining cell annotations 1020a-n. A cell segmentation model 1025 such as a U-Net architecture may be used to generate binary cell segmentation mask images 1030a-n. For example, the cell segmentation model 1025 may be trained on the image patches 1015a-n of original H&E images and their corresponding binary mask images with cell annotations 1020a-n. The annotations may be done manually by pathologists.

After obtaining the binary cell segmentation mask images 1030a-n from the cell segmentation model 1025, the cell segmentations visualization 1035 can be done easily. To visualize the cell boundaries, a binary cell mask (or image from the binary cell segmentation mask images 1030a-n) can be mapped on the corresponding H&E image patch (or image from the image patches 1015a-n) to generate H&E image patch with segmented cells. This exemplary technique as illustrated in FIG. 10, can automate the cell segmentations to reveal the boundaries for the cells on the H&E images and may facilitate multiple downstream tasks, including basic cell counting, performing tumor cellularity calculations, and identifying biomarker.

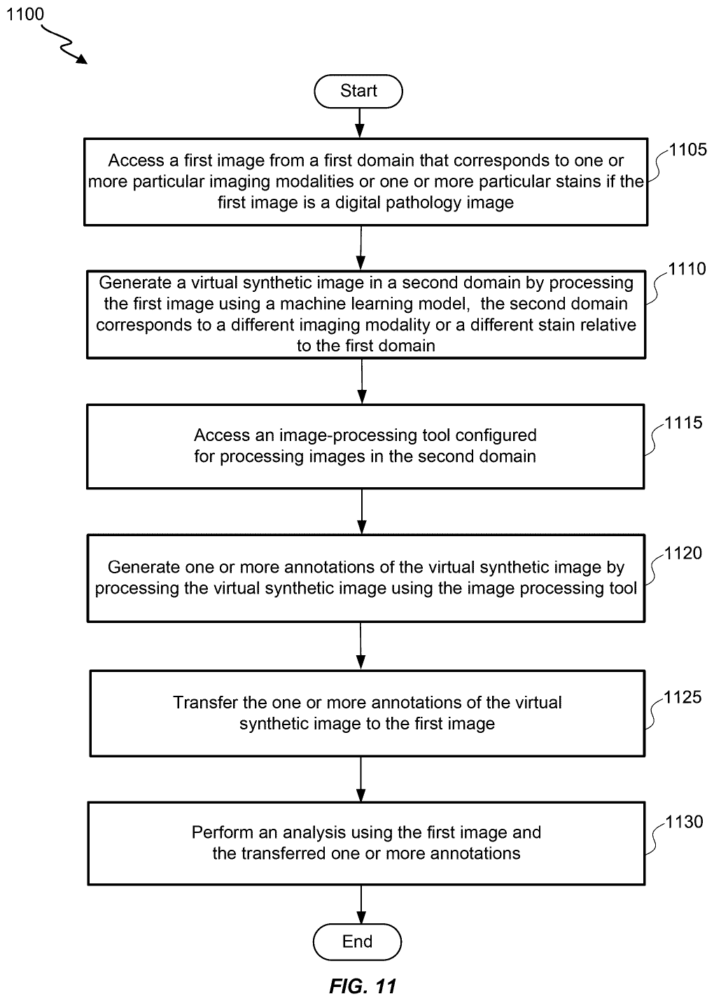

FIG. 11 shows an example flowchart of a system performing domain swap and subsequent processing using existing tools of the second domain. The blocks in flowchart are illustrated in a specific order, while the order can be modified, for example, some blocks may be performed before other, and some blocks may be performed simultaneously. The blocks can be performed by hardware or software or a combination thereof. The process at block 1105 may include accessing a first image from a first domain that corresponds to one or more particular imaging modalities such as multiplex digital pathology image, MPX fluorescence microscopy, MPX brightfield microscopy, MRI, CT, or PET. If the first image is a digital pathology image, then the first domain may correspond to one or more particular stains (e.g., mIHC, mfIHC, H&E). For example, a slide with a slice of a tissue sample can be stained using multiple IHC markers, resulting in multiplex digital pathology image that may be a duplex, a triplex or fourplex image etc. If only one IHC marker is used thereby generating a singleplex image comprising one color/stain.

A virtual synthetic image in a second domain is generated by processing the first image using a machine learning model, at block 1110. The second domain corresponds to a different imaging modality or a different stain relative to the first domain. The machine learning model may be trained on a dataset comprising of images from the first domain and the second domain. An image processing tool can be accessed that is configured to process images in the second domain, at block 1115. In the case of digital pathology images, the image processing tool may include segmentation of tumor, stroma, or cells.

One or more annotations of the virtual synthetic image can be generated by processing the virtual synthetic image using the image processing tool, at block 1120. The one or more annotations may include segmented tumors, stroma, or cells in the virtual synthetic image in second domain. At block 1125, the one or more annotations of the virtual synthetic image can be transferred to the first image of the first domain. Finally, at block 1130, a further analysis may be performed using the first image and the transferred one or more annotations.

### Example

An example implementation of the disclosed technique is provided for domain swap with 6-channel multiplex (mfIHC) as the first domain and the H&E as second domain. More specifically, a section was labeled with six different tags or markers corresponding to biomarkers as mentioned in FIG. 7 to prepare mfIHC sample. Fluorescent microscopy with six channels was used to obtain MPX fluorescence images. The MPX fluorescence images were combined to generate a composite image or the 6-channel multiplex (also referred hereinafter as a multiplex image).

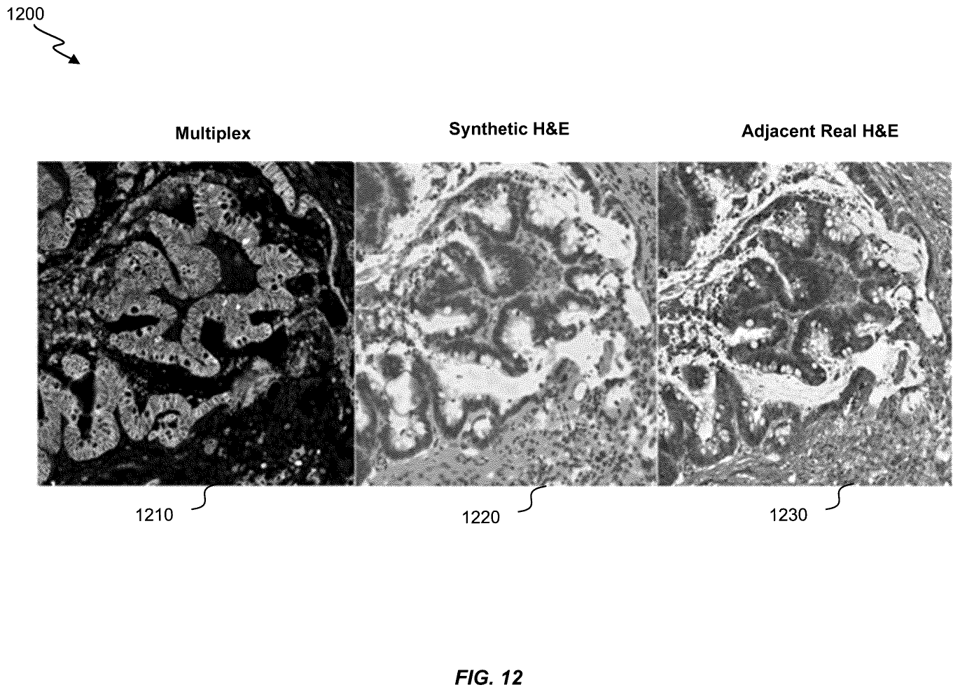

FIG. 12 illustrates a side-by-side comparison of the synthetic H&E image 1220 with the composite image or multiplex image 1210 and adjacent real H&E image 1230, in accordance with an example implementation of the present disclosure. The synthetic H&E image 1220 was generated by using the multiplex image 1210 as the input of the CycleGAN method that is introduced previously in FIG. 6. The multiplex image 1210 represents the composite or multiplex image. The adjacent real H&E image 1230 represents a true H&E image created by pathologists by staining the adjacent slice. The similarities between the synthetic H&E image 1220 and the adjacent real H&E image 1230 provide a degree of confidence of the synthetic results. Importantly, there are high-level conformities between the synthetic H&E image 1220 and the multiplex image 1210 that are stronger than those conformities between the adjacent real H&E image 1230 and the multiplex image 1210. This suggests that the virtual synthetic image could be more valuable than the adjacent slide for reference purposes.

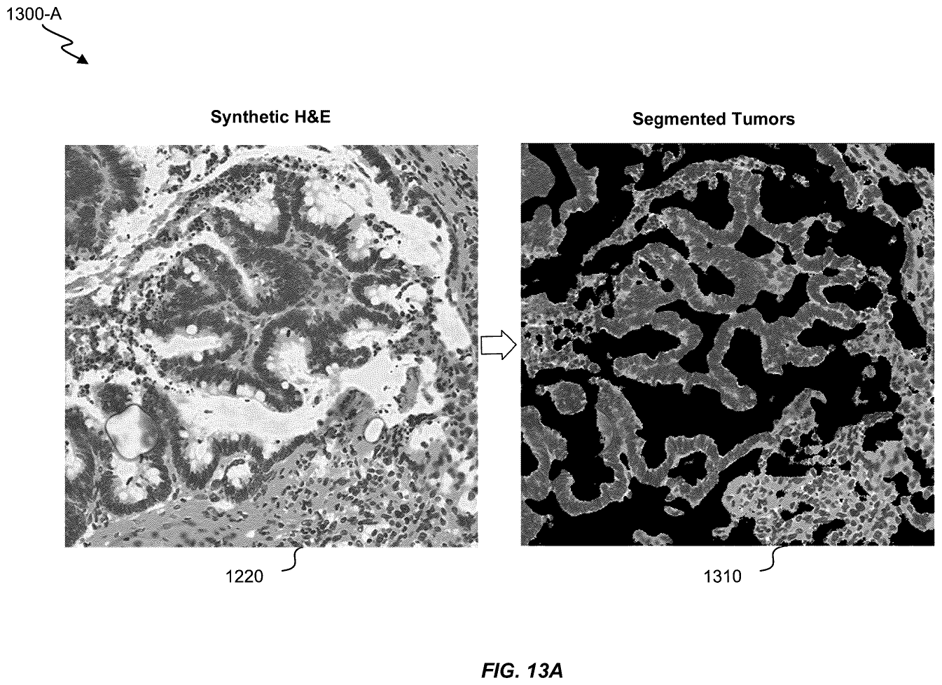

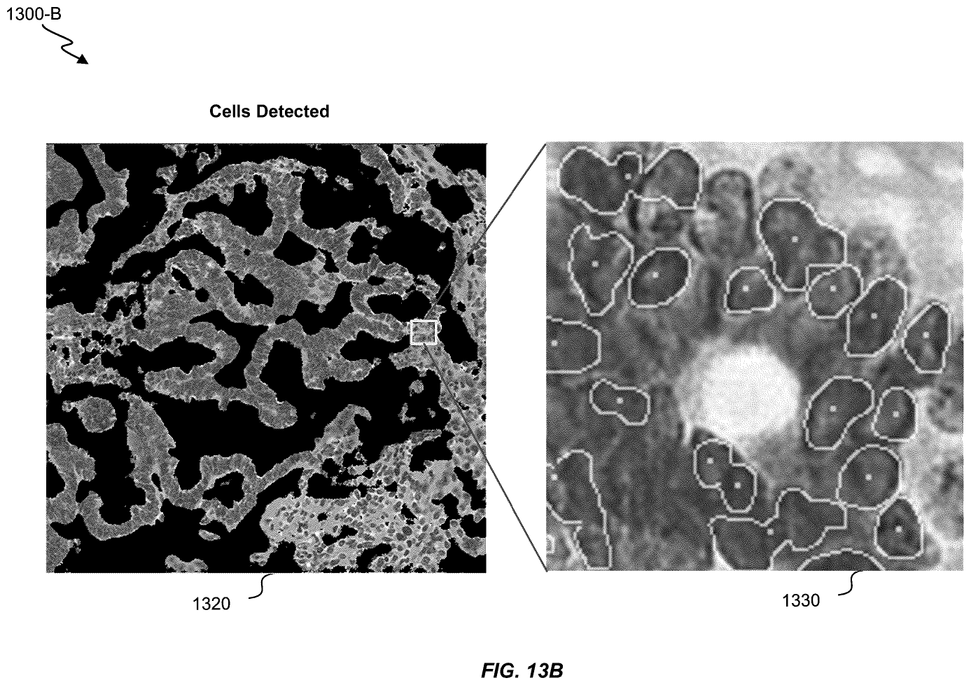

FIG. 13A illustrates a side-by-side comparison of the synthetic H&E image 1220 and segmented tumors 1310 in the synthetic H&E image in accordance with an example implementation of the present disclosure. The segmentation of tumors in the synthetic H&E image 1220 was achieved by utilizing the existing tools developed for H&E domain which are discussed in FIG. 9.

FIG. 13B shows an illustrative example of cells detected 1320 in the synthetic H&E image 1220 with segmented tumors 1310 and a zoomed in patch of cells detected 1330. In the images of cells detected 1320 in segmented tumors 1310, the green circles indicate segmented boundaries of tumor cells, and the green dots indicate predicted tumor nuclei.

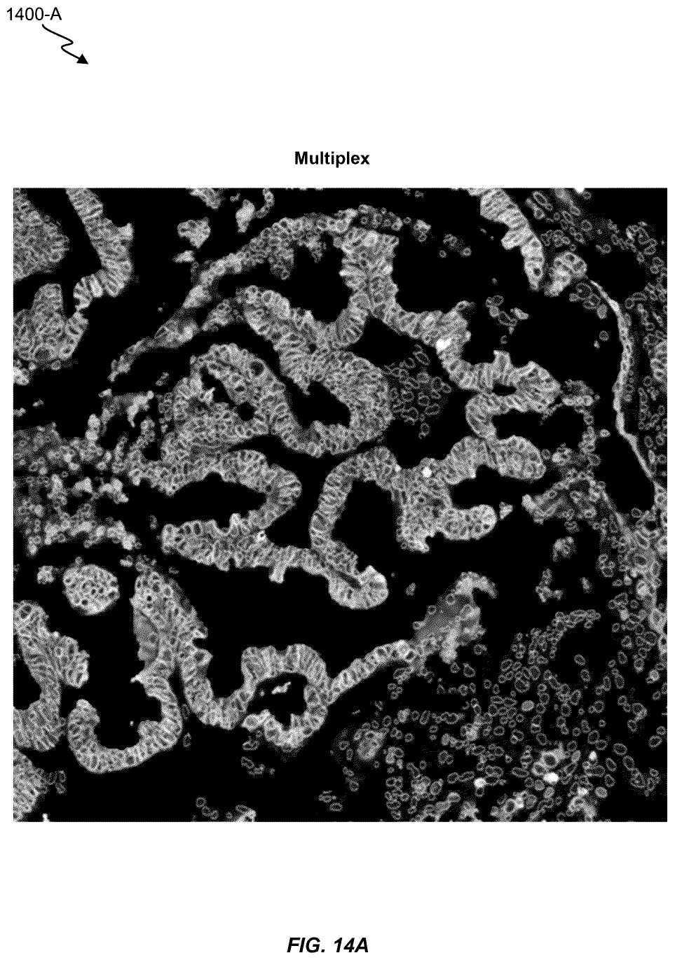

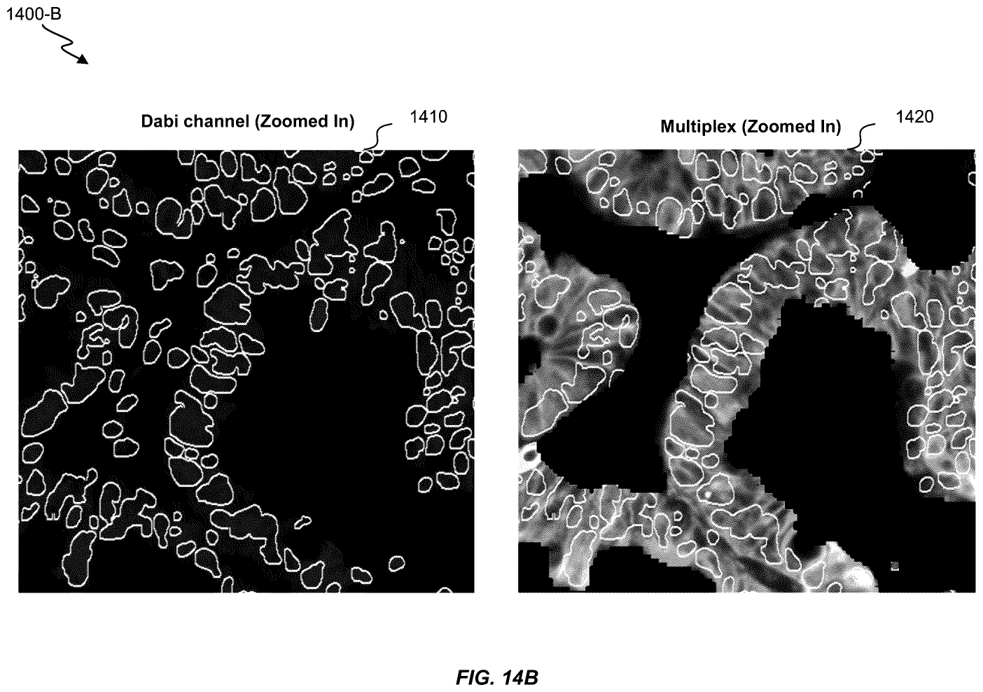

FIG. 14A shows an illustrative example of the composite or multiplex image or the composite image with segmented tumor and cells, in accordance with an example implementation of the present disclosure. The segmentations of the tumors and cells, as identified in the H&E domain by processing the synthetic H&E image 1220, were mapped to the multiplex domain to generate the composite or multiplex image as shown in FIG. 14A. The multiplex image with segmented tumor and cells information can be analyzed further to determine specific problems or diseases.

FIG. 14B shows a zoomed in patch of the composite or multiplex image of FIG. 14A that was segmented using the H&E domain analysis results. The left image shows a zoomed in patch with nuclei segmented results in a given channel (e.g., a dapi channel 1410) of the multiplex image. Similarly, the right image represents a zoomed in patch of the multiplex image 1420 with segmented results transferred from the H&E domain. These figures illustrate that the boundaries identified by processing the virtual synthetic H&E image appear to map well to the multiplex domain.

Thus, various techniques disclosed in the present disclosure can be utilized for transferring existing tools or models to different imaging modalities or domains. As in the above example, the synthetic virtual H&E image was generated from the multiplex image by using CycleGAN. This synthetic virtual H&E image can act as a bridge (domain swap) and can eliminate the requirement of staining (e.g., H&E) and processing of sequential slices while saving resources (e.g., time, cost, labor). Through synthetic virtual H&E images, the multiplex domain is transferred into H&E domain. Afterwards, leveraging tools or deep learning algorithms previously developed in the H&E domain may facilitate segmentation of tumors, and/or stroma tissues, artifacts detection, or the cell segmentations to nicely draw the boundaries for the cells on the H&Es. The disclosed approach boost precision and expedites multiplex algorithm development, by transferring results such as the tumor map and cell segmentations in the multiplex domain. Since the spatial reference points are the same, results, annotations, detections, or segmentations in the H&E domain can be transferred seamlessly to multiplex domain.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A computer-implemented method comprising:
accessing a first image from a first domain, wherein the first domain corresponds to one or more particular imaging modalities, and wherein the first domain further corresponds to one or more particular stains if the first image is a digital pathology image;
generating a virtual synthetic image by processing the first image using a machine learning model, wherein the virtual synthetic image is in a second domain that corresponds to a different imaging modality or a different stain relative to the first domain;
accessing an image-processing tool configured for processing images in the second domain;
generating one or more annotations of the virtual synthetic image by processing the virtual synthetic image using the image-processing tool;
transferring the one or more annotations of the virtual synthetic image to the first image; and
performing an analysis using the first image and the transferred one or more annotations.

2. The method of claim 1, wherein the machine learning model comprises a cycleGAN.

3. The method of claim 1, wherein the first domain is darkfield multiplex and the second domain is H&E.

4. The method of claim 1, wherein the first domain is immunohistochemistry and the second domain is H&E.

5. The method of claim 1, wherein the first domain or the second domain is magnetic resonance imaging.

6. The method of claim 1, wherein the one or more annotations include an annotation of a tumor, stroma or artifact region.

7. The method of claim 1, wherein the one or more annotations include a segmentation of each of a set of cells.

8. A system comprising:
one or more data processors; and
a non-transitory computer readable storage medium containing instructions which, when executed on the one or more data processors, cause the one or more data processors to perform a set of operations including:
accessing a first image from a first domain, wherein the first domain corresponds to one or more particular imaging modalities, and wherein the first domain further corresponds to one or more particular stains if the first image is a digital pathology image;
generating a virtual synthetic image by processing the first image using a machine learning model, wherein the virtual synthetic image is in a second domain that corresponds to a different imaging modality or a different stain relative to the first domain;
accessing an image-processing tool configured for processing images in the second domain;
generating one or more annotations of the virtual synthetic image by processing the virtual synthetic image using the image-processing tool;
transferring the one or more annotations of the virtual synthetic image to the first image; and
performing an analysis using the first image and the transferred one or more annotations.

9. The system of claim 8, wherein the machine learning model comprises a cycleGAN.

10. The system of claim 8, wherein the first domain is darkfield multiplex and the second domain is H&E.

11. The system of claim 8, wherein the first domain is immunohistochemistry and the second domain is H&E.

12. The system of claim 8, wherein the first domain or the second domain is magnetic resonance imaging.

13. The system of claim 8, wherein the one or more annotations include an annotation of a tumor, stroma or artifact region.

14. The system of claim 8, wherein the one or more annotations include a segmentation of each of a set of cells.

15. A computer-program product tangibly embodied in a non-transitory machine-readable storage medium, including instructions configured to cause one or more data processors to perform a set of operations comprising:
accessing a first image from a first domain, wherein the first domain corresponds to one or more particular imaging modalities, and wherein the first domain further corresponds to one or more particular stains if the first image is a digital pathology image;
generating a virtual synthetic image by processing the first image using a machine learning model, wherein the virtual synthetic image is in a second domain that corresponds to a different imaging modality or a different stain relative to the first domain;
accessing an image-processing tool configured for processing images in the second domain;
generating one or more annotations of the virtual synthetic image by processing the virtual synthetic image using the image-processing tool;
transferring the one or more annotations of the virtual synthetic image to the first image; and
performing an analysis using the first image and the transferred one or more annotations.

16. The computer-program product of claim 15, wherein the machine learning model comprises a cycleGAN.

17. The computer-program product of claim 15, wherein the first domain is darkfield multiplex and the second domain is H&E.

18. The computer-program product of claim 15, wherein the first domain is immunohistochemistry and the second domain is H&E.

19. The computer-program product of claim 15, wherein the first domain or the second domain is magnetic resonance imaging.

20. The computer-program product of claim 15, wherein the one or more annotations include an annotation of a tumor, stroma or artifact region.
